(19) United States
(12) Patent Application Publication
(10) Pub. No.: US 2007/0203456 A1
(43) Pub. Date: Aug. 30, 2007

(54) Percutaneous surgical instrument system and method

(76) Inventor: W. Thomas McClellan, Fort Lauderdale, FL (US)

Correspondence Address: LERNER GREENBERG STEMER LLP, P O BOX 2480, HOLLYWOOD, FL, 33022-2480, US

(21) Appl. No.: 11/361,421

(22) Filed: Feb. 24, 2006

(51) Int. Cl. A61M 5/178

(52) U.S. Cl. 604/168.01

(57) ABSTRACT

A percutaneous surgical instrument system includes a handle to be manipulated by an operator, a carrier and a trocar to be manipulated by the handle. The trocar is inserted through the carrier for penetration to a surgical site and removed from the carrier to permit insertion of a surgical instrument through the carrier. A syringe supplies fluid to the trocar during penetration to the surgical site. A method for percutaneously inserting a surgical instrument to a surgical site is also provided.

### BACKGROUND OF THE INVENTION

1. Field of the Invention

The invention relates to a percutaneous surgical instrument system and method. A carrier and its trocar are used as an insertion aid and access portal for percutaneous surgical instruments, such as chest tubes, percutaneous abdominal instrumentations, biopsy needles, endoscopes and laparoscopes.

2. Description of the Related Art

Percutaneous surgical instrument carriers, with their pointed lumen-filling trocars, must penetrate several tissue layers to reach the intended operative or diagnostic site. The carrier, with its insertion trocar removed, is a tube-like portal or sleeve through which the surgical instrument functions internally. The carrier and trocar are inserted as a single unit, and then the carrier with its selected surgical instrument are used operatively as a single unit.

Such relatively large-bore carriers, having the tapered and pointed central lumen filler or trocar to aid insertion, meet with exaggerated resistance related to a phenomenon of pressure desiccation or pressure drying of the invaded tissue. The advancement force or pressure of the large bore insertions, forces the normally present tissue fluids away from the invading path, and in-effect dry the tissue and increase insertion resistance. Furthermore, the advancement of the trocar and carrier stretches and tightens the encircling fibers of the tissue layers causing a gripping resistance much like a Chinese finger trap. That elevated resistance depresses, distorts, or tents downward the impinged tissue layers and impairs the controlled safe advancement of the carrier and trocar. More importantly, the operators' modulating proprioceptive feedback or feel of proper advancement is prevented. That elevated insertion force increases the risk of sudden break-through or over-insertion and collateral damage to nearby organ systems and the downward tenting of the tissue layers or abdominal wall brings the sharpened trocar unsafely close to nearby organ systems.

Such devices, which suffer from the disadvantages described above, are known in the prior art from U.S. Pat. No. 4,972,827 to Kishi et al., U.S. Pat. No. 5,059,186 to Yamamoto et al., U.S. Pat. No. 5,368,574 to Antonacci et al., U.S. Pat. No. 4,897,081 to Poirier et al. and U.S. Pat. No. 5,626,597 to Urban et al.

### SUMMARY OF THE INVENTION

It is accordingly an object of the invention to provide a percutaneous surgical instrument system and method, which overcome the hereinafore-mentioned disadvantages of the heretofore-known systems and methods of this general type and which are safer and have a low insertion resistance.

With the foregoing and other objects in view there is provided, in accordance with the invention, a percutaneous surgical instrument system. The system comprises a handle to be manipulated by an operator and a carrier. A trocar is to be manipulated by the handle, inserted through the carrier for penetration to a surgical site, and removed from the carrier to permit insertion of a surgical instrument through the carrier. A syringe is provided for supplying fluid to the trocar during penetration to the surgical site.

In accordance with another feature of the invention, the trocar has a tip through which the syringe supplies the fluid to the surgical site. The tip has at least one opening formed therein for supplying the fluid to the surgical site. The tip has an outer periphery with channels formed therein for directing the fluid, and the at least one opening is disposed in at least one of the channels. The channels extend substantially in axial direction of the trocar and are spaced apart circumferentially entirely over the outer periphery of the tip. The carrier has an outer periphery with channels formed therein for directing the fluid along with the channels formed in the tip of the trocar.

According to the invention, the fluid and channels provide a pressure-limited, lubricated carrier and trocar with reduced contact area, to safely reduce percutaneous insertion resistance, improve operator feed back and control, reduce tissue damage, lessen post operative discomfort and reduce the risk of collateral organ damage during percutaneous instrument carrier insertion.

In accordance with a further feature of the invention, the carrier has a widened proximal entrance for receiving the trocar or surgical instrument and a distal opening with a sharpened edge. The widened proximal entrance facilitates insertion of the trocar or surgical instrument. The sharpened edge aids insertion into subject tissue at the surgical site.

In accordance with an added feature of the invention, the handle has a bore formed therein for at least partly receiving the syringe and at least one passageway leading to a passageway in the trocar. The syringe supplies the fluid through the at least one passageway in the handle and the passageway in the trocar to the at least one opening in the tip of the trocar.

In accordance with an additional feature of the invention, the handle has a threaded nipple and a threaded nut. The nipple and nut connect the at least one passageway in the handle through the nipple to the passageway in the trocar.

In accordance with yet another feature of the invention, the syringe has a plunger and a body with a cylindrical container for receiving the plunger. The plunger is manually depressed for injecting the fluid into the handle. The plunger has a head with a recess formed therein and a projection disposed in the recess. The recess and projection form a ring seal with play allowing the fluid to leak out for limiting and controlling injection pressure while adequately lubricating the carrier.

With the objects of the invention in view, there is also provided a method for percutaneously inserting a surgical instrument to a surgical site. The method comprises inserting a trocar through a carrier. The carrier and the trocar are inserted to the surgical site while guiding a fluid through the trocar. The trocar is removed from the carrier and the surgical instrument is inserted through the carrier.

In accordance with another mode of the invention, the fluid is directed through at least one opening in at least one channel in an outer periphery of a tip of the trocar. The channels extend substantially in axial direction of the trocar and are spaced apart circumferentially entirely over the outer periphery of the tip. The fluid is directed along the channels formed in the tip of the trocar and along channels formed in an outer periphery of the carrier. Thus, the lengths of the trocar and carrier are lubricated during insertion.

In accordance with a concomitant mode of the invention, the fluid is supplied from a syringe through a handle to be manipulated by an operator and through the trocar to the at least one opening during penetration to the surgical site. The fluid is allowed to leak out of a ring seal in the syringe for limiting and controlling injection pressure while adequately lubricating the carrier. It is therefore ensured that the pressure of the fluid from the syringe to the tip of the trocar cannot exceed safe limits.

Other features which are considered as characteristic for the invention are set forth in the appended claims.

Although the invention is illustrated and described herein as embodied in a percutaneous surgical instrument system and method, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE DRAWINGS

FIG. 1 is a diagrammatic, side-elevational view of a handle, trocar, carrier and syringe of a percutaneous surgical instrument carrier and trocar system according to the invention;

FIG. 2 is a perspective view, similar to FIG. 1, of the system according to the invention;

FIG. 3 is an exploded, side-elevational view of the handle and nut of the system according to the invention;

FIG. 4 is a view similar to FIG. 3, but in which the handle and nut are transparent to reveal internal features;

FIG. 5 is an exploded, side-elevational view of a plunger and body of the syringe of the system according to the invention;

FIG. 6 is a perspective view of the syringe, similar to FIG. 5;

FIG. 7 is a longitudinal-sectional view of the syringe of FIGS. 5 and 6;

FIG. 8 is a perspective, longitudinal-sectional view of the syringe of FIGS. 5, 6 and 7;

FIG. 9 is a side-elevational view of the carrier of the system according to the invention;

FIG. 10 is a fragmentary, enlarged, side-elevational view of the carrier showing its fluting;

FIG. 11 is a perspective view of the carrier similar to FIG. 10;

FIG. 12 is a perspective view of the trocar of the system according to the invention;

FIG. 13 is a fragmentary, enlarged, side-elevational view of the trocar showing its fluting;

FIG. 14 is a fragmentary, side-elevational view of the tip of the trocar within the carrier;

FIG. 15 is an exploded, side-elevational view, prior to assembly, of the syringe, handle, trocar and carrier of the system according to the invention;

FIG. 16 is a perspective view similar to FIG. 15;

FIG. 17 is a side-elevational view, after assembly, of the syringe, handle and carrier of the system according to the invention; and

FIG. 18 is a perspective view similar to FIG. 17.

### DESCRIPTION OF THE PREFERRED EMBODIMENTS

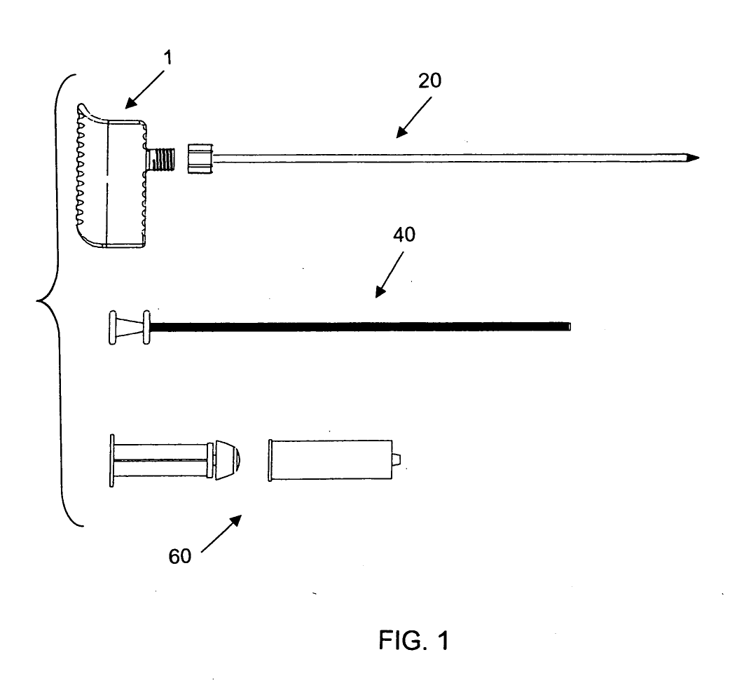

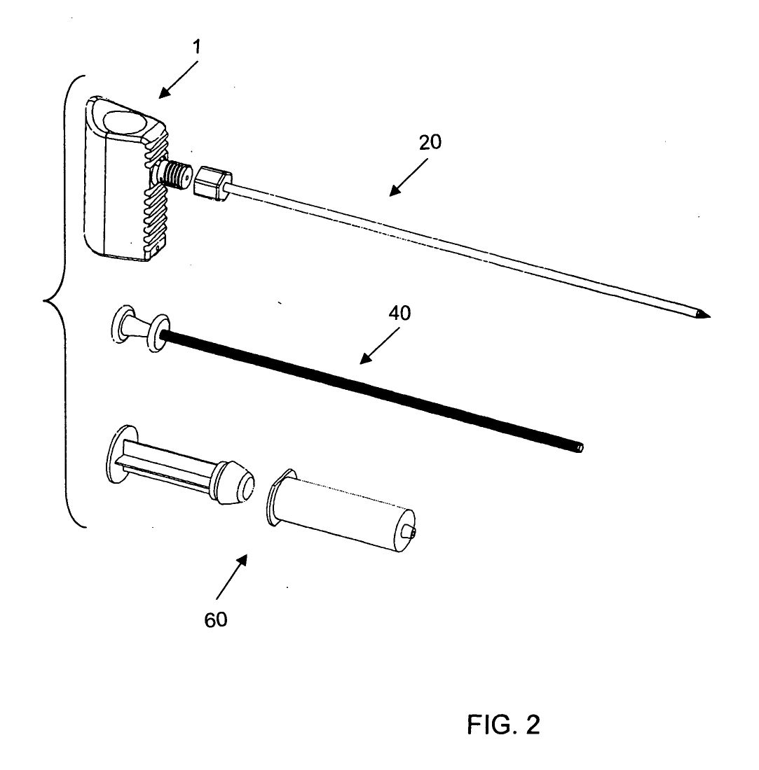

Referring now to the figures of the drawings in detail and first, particularly, to FIGS. 1 and 2 thereof, there is seen a percutaneous surgical instrument carrier and trocar system according to the invention, including a handle 1, a trocar 20, a carrier 40 and a syringe 60.

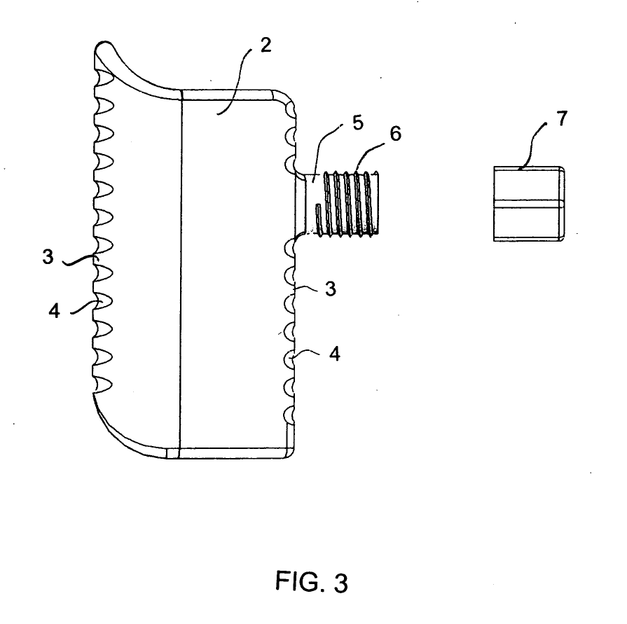

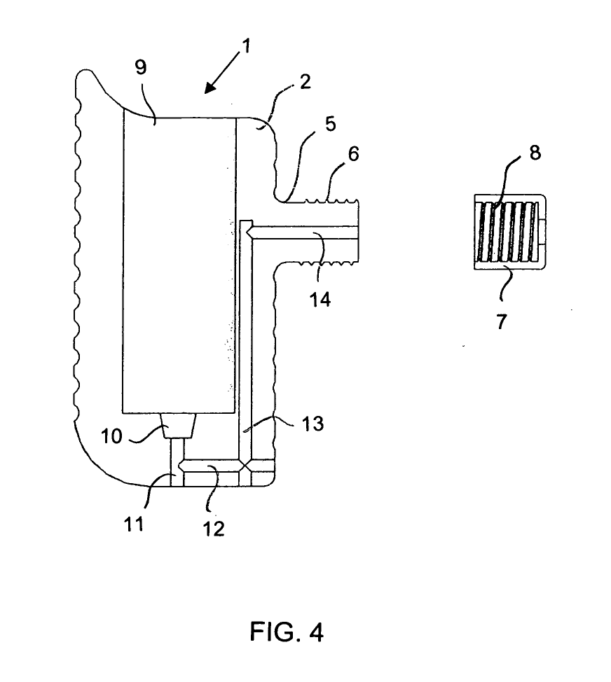

As is seen in FIG. 3, the handle 1 has a body 2 with ridges 3 and recesses 4 to facilitate gripping by an operator at the proximal end of the system. It can also be seen that the body 2 has a nipple 5 with external screw threads 6. An adjusting nut 7 has internal screw threads 8 shown in FIG. 4 for mating with the external threads 6. FIG. 4 additionally shows that a cylindrical container-receiving bore 9 leads to a tapered nozzle-receiving bore 10, formed in the body 2. Channels or passageways 11, 12, 13 and 14 lead in succession from the bore 10 to the nipple 5. The channels 11, 12, 13 are closed where they reach the outer surface of the handle.

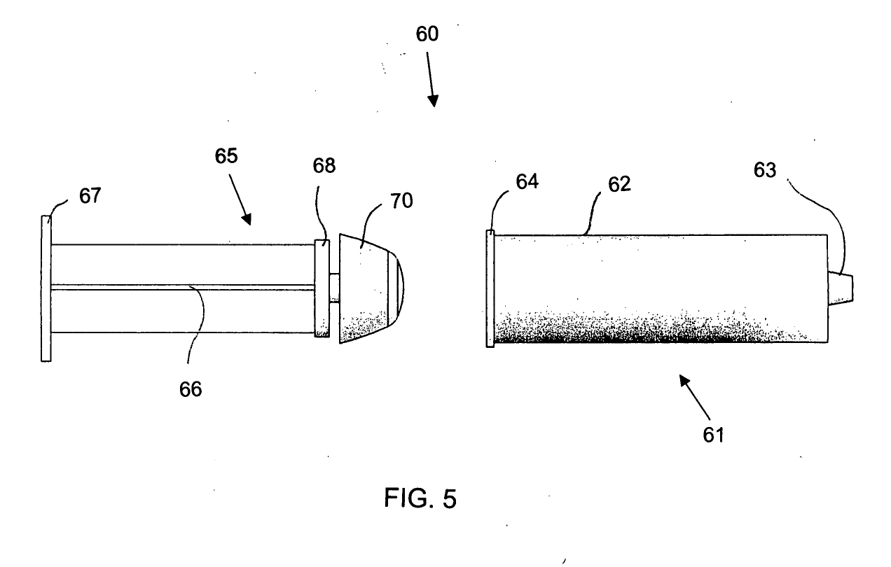

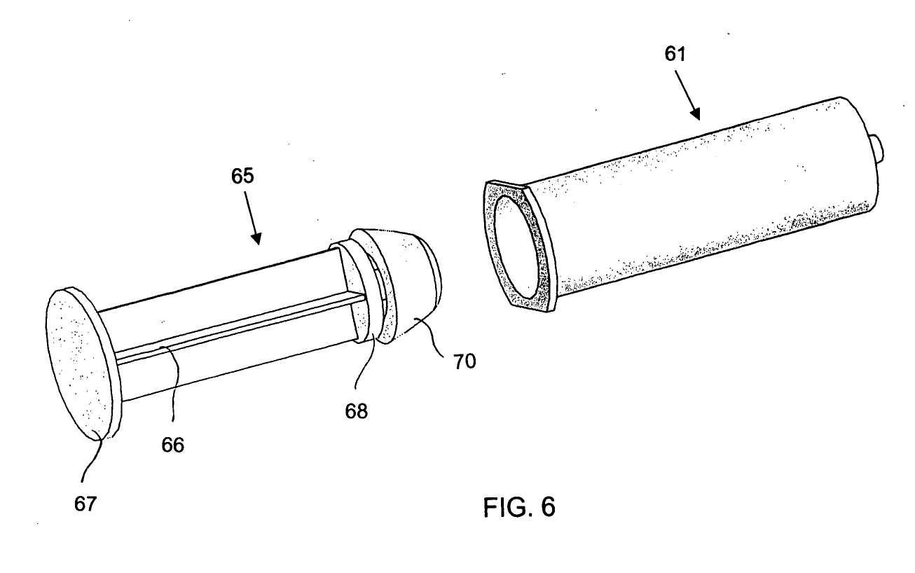

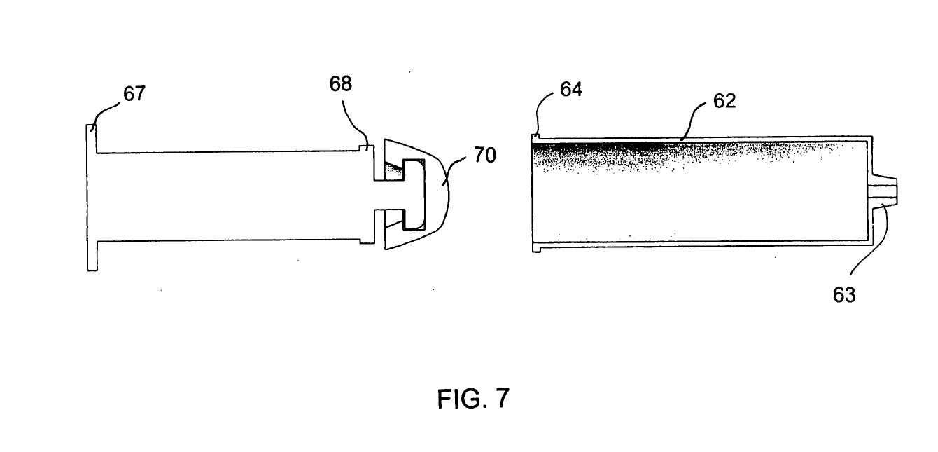

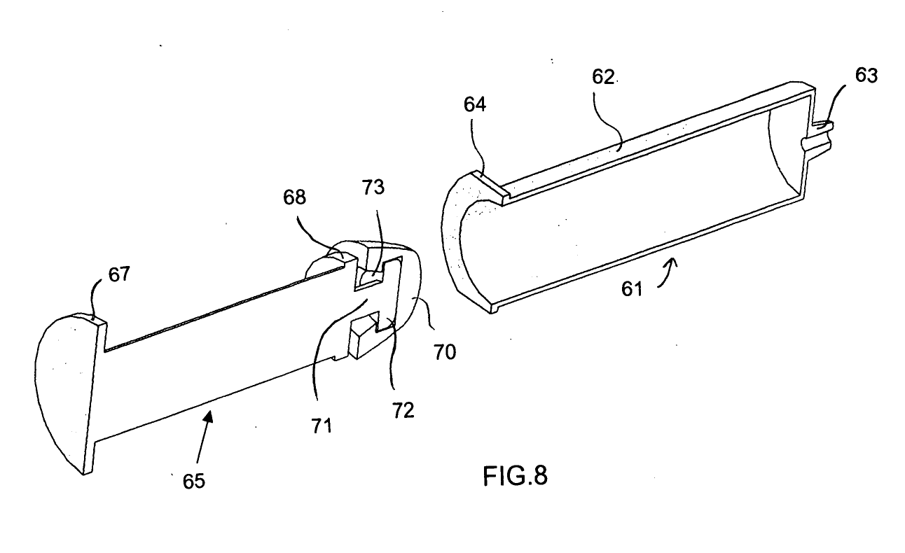

The syringe 60 shown in FIGS. 5, 6, 7 and 8 has a body 61 and a plunger 65. The body 61 has a cylindrical container 62 and a tapered nozzle 63 which correspond in size and shape to the cylindrical bore 9 and the tapered bore 10 of the handle 1. The body 61 also has a lip 64 with a periphery having flattened portions, as is seen in FIGS. 6 and 8. The plunger 65 has ribs 66 extending between a disk 67 to be pressed by the thumb of an operator of the system and a disk 68 in the vicinity of a head 70 of the plunger 65. A T-shaped projection at the end of the plunger 65 has a shaft 71 and a crosspiece 72. The head 70 of the plunger 65 has a mushroom-shaped recess 73 formed therein for receiving the shaft 71 and the crosspiece 72 of the T-shaped projection.

The cylindrical container 62 of the pressure-limiting syringe 60 is pre-filled with saline or other acceptable lubricant and the tapered nozzle 63 is secured to the tapered nozzle-receiving bore 10 at the bottom of the protected, cylindrical, container-receiving bore 9 in the handle 1.

The projection 71, 72 and the recess 73 of the lubricating syringe 60 provide a unique reverse-angle or with-the-flow ring seal, configured to intentionally bypass or leak fluid at pressures exceeding the average human systolic pressure of 120 mm of mercury and cause over-pressure release or venting, along the side of the plunger 65. It is noted that conventional syringes have seals that face against-the-flow, or are configured to expand and seal more firmly with increasing pressure.

By limiting and controlling the injection pressure, adequate lubrication of the advancing carrier is permitted without unnecessary lubricant extravasation. The over-pressure release or venting, along the side of the syringe plunger is easily felt by the operator for better modulation and control. The reverse-angled syringe seal vents excess or harmful pressure, while still allowing effective lubrication, and protection of tissue. The seal material is manufactured in a strength and contour to yield predictably at design pressures.

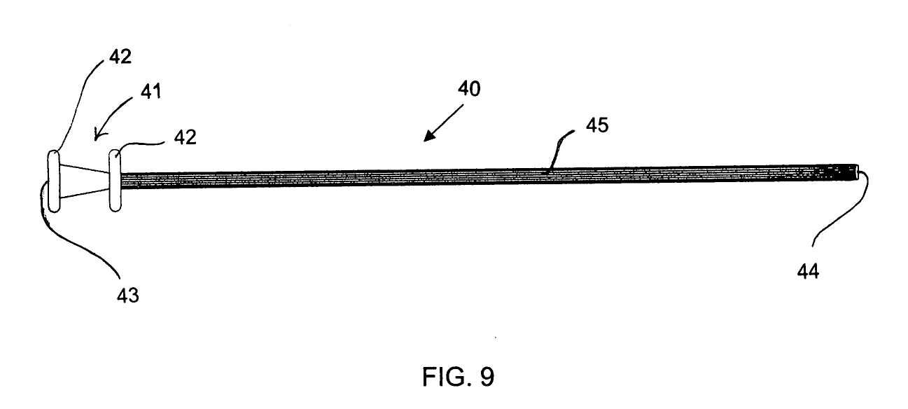

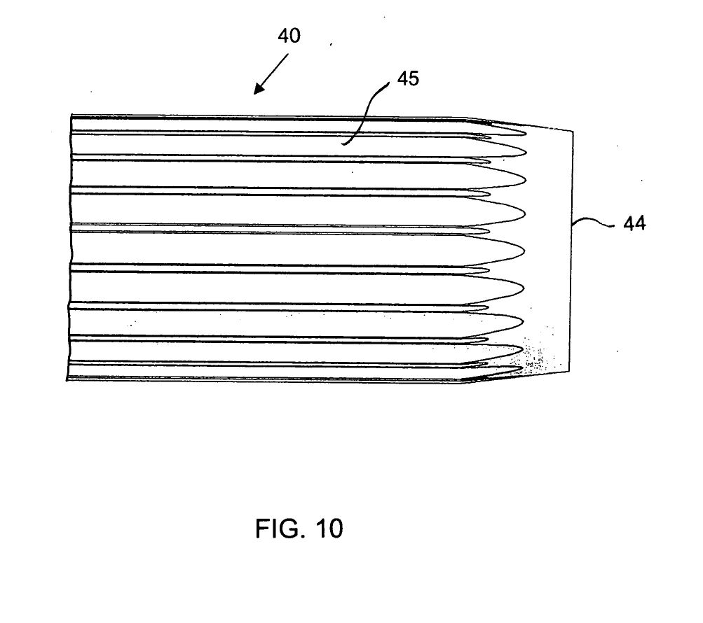

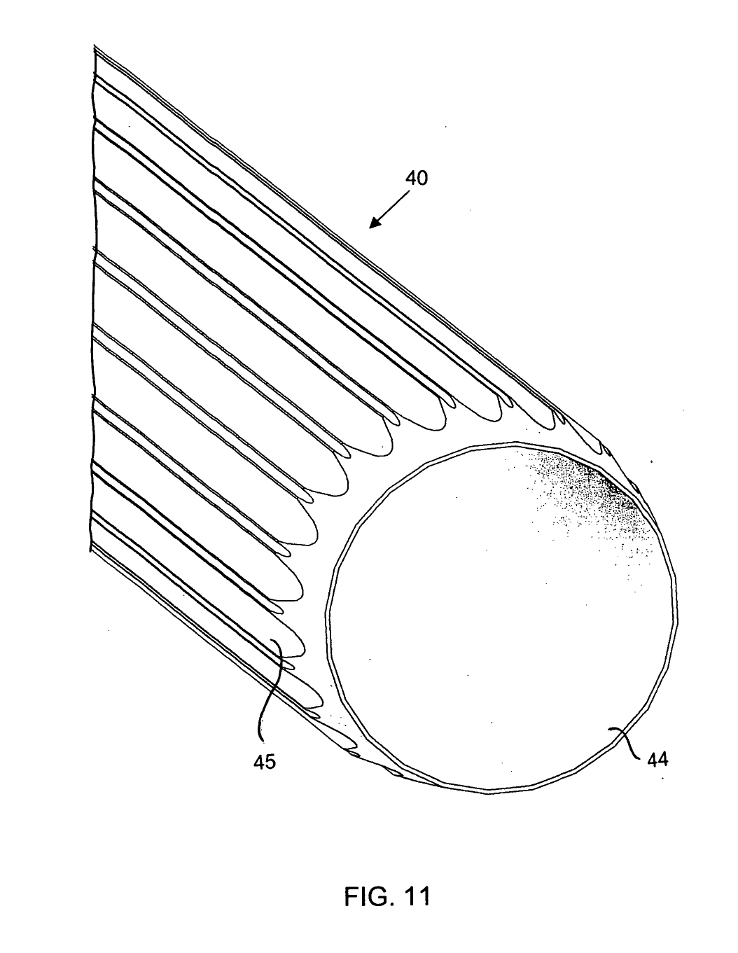

As is seen in FIG. 9, the carrier 40 has a handle 41 at a proximal end. The handle 41 has at least one control ring 42 and a conically-tapered entrance 43 for a trocar or surgical instrument. The entrance 43 of the carrier 40 is conically shaped or tapered to the lumen of the carrier at the proximal end for facilitating the introduction of the surgical instrument and trocar 20 without abrasion, damage or dulling of their sharpened edges. The carrier 40 also has an opening 44 at a distal end. The opening 44 of the tube-like surgical instrument and trocar carrier 40 is tapered to facilitate and protect the insertion of the trocar 20 or surgical instrument. The opening 44 at the distal end has a right angle or straight across termination, which is tapered and has a sharpened edge around its entire circumference as is best seen in FIGS. 10 and 11, to aid insertion into the subject tissue. A passageway extends within the carrier 40 from the entrance 43 to the opening 44. As is seen particularly well in FIGS. 10 and 11, fluted or concave channels 45 are provided around the entire circumference of the carrier 40 from the distal-most control ring 42 to the opening 44.

The carrier 40 is constructed of metal or other suitable material, in matching variations of lengths from 8 cm to 40 cm and in equivalent cross-sectional diameters of 10 to 18 gauge. The carrier 40 has cross-sectional shapes matching the surgical instruments and trocars 20, but is slightly larger, since the carrier is constructed to fit snuggly over the surgical instrument or trocar, for ease of introduction through the body layers and to safely contain and support the surgical instrument to the proper location and anglulation through the carrier 40.

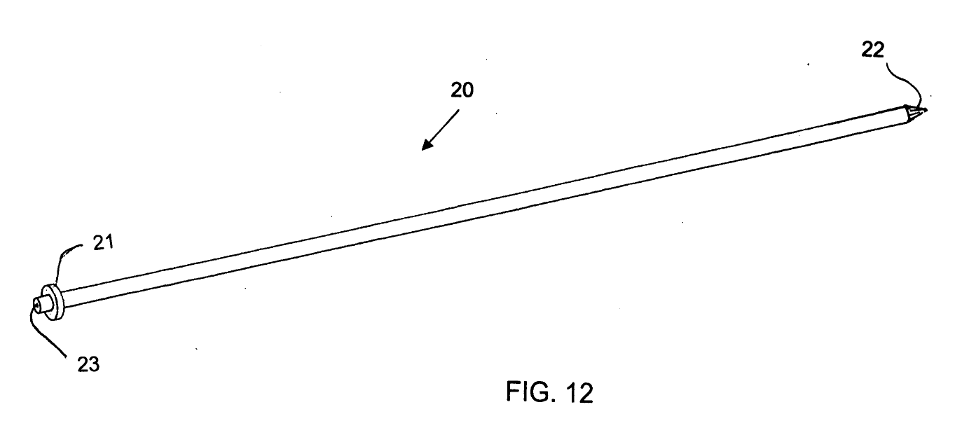

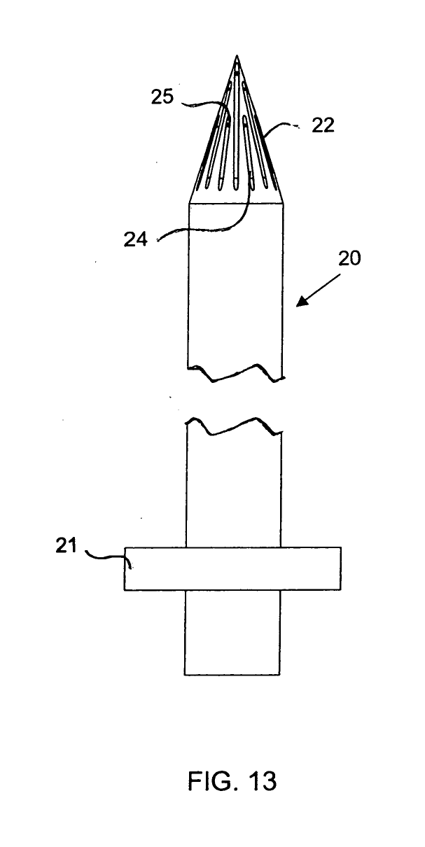

The trocar 20 shown in FIGS. 1, 2, 12 and 13 has a gripping handle 21 at a proximal end and a tip 22 at a distal end. A continuous passageway 23 leads from the handle 21 to the tip 22. As is seen in FIG. 13, the tip 22 has a tapered and sharpened point with fluted or concave channels 24 around its entire circumference. One or more fluid outlets 25 are formed within the channels 24. The injected lubricant fluid or saline is guided through the passageway 23 and evenly from the outlets 25 along all areas.

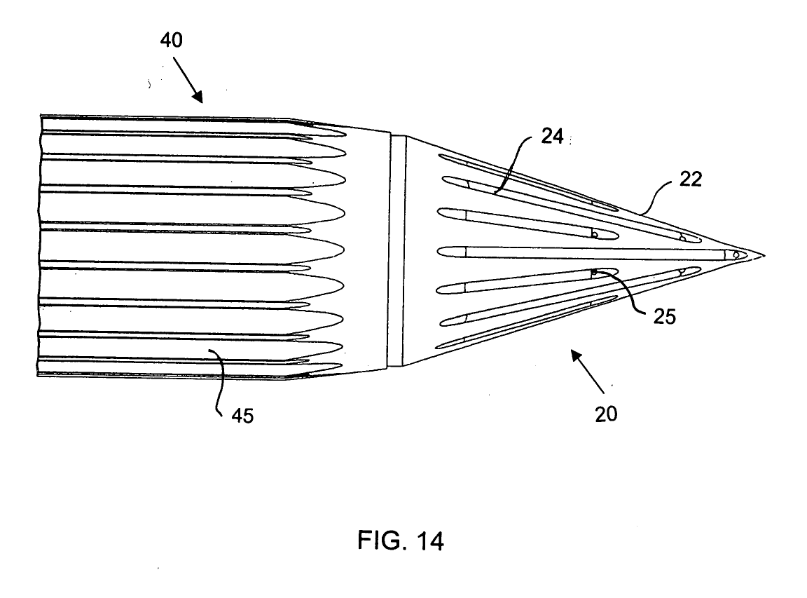

FIG. 14 also shows that the tapered and sharpened point at the tip 22 of the trocar 20 extends beyond the carrier 40. The protruding tapered and sharpened tip 22 is constructed with the fluted surface of concave channels 24 around its entire circumference, running in line with and straight back from the tip to the carrier 40 and matching the fluted or concave channels 45 of the forward portion of the carrier. The fluted or concave channels 24 around the circumference of the trocar lead the injected lubricant fluid or saline from the tip of the trocar to the carrier and along the matching and similarly fluted or concave channels 45 of the carrier. These fluted or concave channels reduce the actual contact surface area, since the fibers touch only the very tips of each concave ridge, and additionally channel the remaining interstitial fluid evenly along the sides of the trocar 20 and carrier 40 for further reduction of insertion resistance.

The fluted or concave channels 45, 24 of the carrier 40 and the tip 22 of the trocar 20 facilitate insertion and penetration of high density, high fiber layers. The channels 45, 24 of the high-density, high-fiber layer penetrating trocar tip and carrier improve lubrication and reduce contact surface and tissue resistance from pressure desiccation. The concave channel shapes also increase reflectivity and visibility by all imaging systems for better guidance and location control. The fluted or faceted channels reflect ultrasound, X-ray and echoed MRI energy more efficiently, which increases the visibility of the carrier and trocar by remote imaging systems, for improved control and placement.

Insertion of the relatively large bore round object through multiple fibrous layers meets with an exaggerated resistance related to the phenomenon of pressure desiccation or drying and stretching by the compression of tissue layers ahead of the trocar 20 and carrier 40. The forced advancement of the encased round trocar 20 drives the normal interstitial fluid from the contacting tissue and stretches the fibrous layers, creating a collapsing, tightening, fibrous tube surrounding the carrier 40 and trocar 20, much like a Chinese finger trap. This pronounced increase in resistance takes significant pressure to overcome, decreases the tactile feel or proprioceptive feedback to the operator and increases the risk of misplacement or break-through-over-insertion trauma to other organ systems.

The gripping resistance of the constricting tissue is reduced by the unique effect of a reduced contact or grip-able surface area created by the contour of the fluted or concave channels 24, 45 of the tip 22 of the trocar 20 and the carrier 40. The stretched and tightened encircling fiber bands are pulled into straight line fibers, therefore contacting only elevated ridges between the fluted channels. Surface contact area and encircling fiber resistance is markedly reduced. Additionally, the lubricating fluid flowing through the fluted concave channels 24, 45 prevents the tensioned tissue from touching or resisting the remaining non-contact surface area of the trocar or carrier. By safely injecting sterile normal saline at limited pressures, ahead of and along the body of the trocar and carrier during insertion, and by markedly reducing the actual surface contact area, normal tissue lubrication is preserved, resistance and tissue damage is reduced, post operative discomfort is lessened, proprioceptive feedback and control are returned, and the risk of unintentional over insertion collateral damage is reduced.

The trocar 20 and carrier 40 are inserted and maneuvered as a single unit. Between uses of the surgical instrument, such as for biopsies, the trocar 20 is reinserted within the carrier 40 for any repositioning or angle change of the carrier. The objective is to insert and maneuver the carrier 40 to the perfect depth and position. The carrier 50 may also be constructed with etched markings of insertion length, in centimeters, on its outer lateral surfaces for more precise placement.

The trocar 20 and carrier 40 are configured and manufactured of suitable material, in various sizes, lengths and shapes to support and fit the intended surgical instruments. However, the carrier 40 is intentionally shorter than the surgical instruments, such as by four centimeters, providing for precise adjustability of depth in 1 centimeter increments, from 4 cm down to 1 cm in length. It should be noted that these dimensions are given as examples only and are not intended to be limiting.

The trocar 20 acts as an insertion and strengthening aid for the carrier 40. In percutaneous biopsy, for example, there are several layers of tissue which a biopsy needle must pass through, in order to reach a biopsy site and since such relatively large bore needles cannot be made with cutting tips because of tissue damage, a tapered and pointed central lumen filler, or trocar 20 with a gripping handle or control ring 21, must be added to assist insertion. The biopsy needle or other surgical instrument is also relatively long and is maneuvered during insertion with bends and angle changes. These leverages or bending forces also require the stabilizing and strengthening of the carrier 40 with a full size strong trocar 20. The trocar 20 is constructed to fit snuggly within the lumen of the carrier 40, matching the carrier in size, cross-sectional shape and being slightly longer in length with a finely tapered point at the tip 22 that protrudes from the carrier 40 as is seen in FIG. 14.

After the trocar and carrier have been inserted to the desired location, the trocar is removed, leaving the carrier to guide a surgical instrument. The entrance 43 at the proximal end of the carrier 40 is adapted to fit the trocar 20 and the various intended surgical instruments.

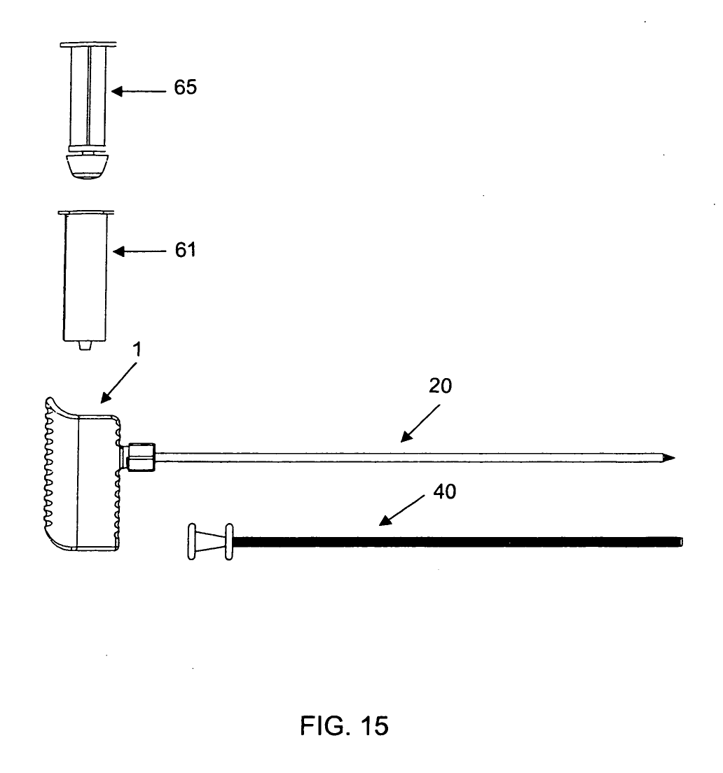

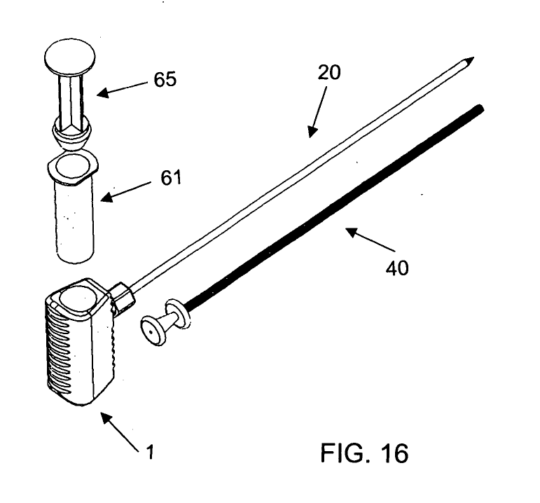

FIGS. 15 and 16 show the body 61 and the plunger 65 of the syringe 60, as well as the handle 1, the trocar 20 and the carrier 40 immediately before assembly. However, the adjusting nut 7 has been slid over the tip 22 of the trocar 20 to the gripping handle 21 and the nut 7 has then been screwed onto the nipple 5.

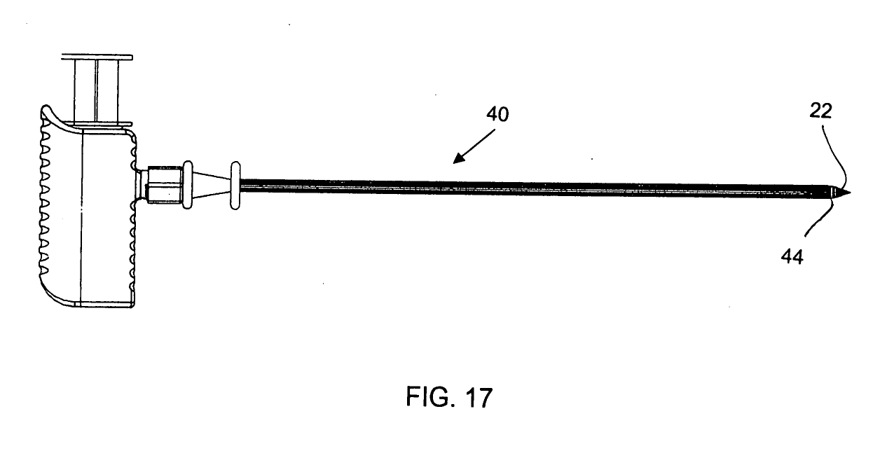

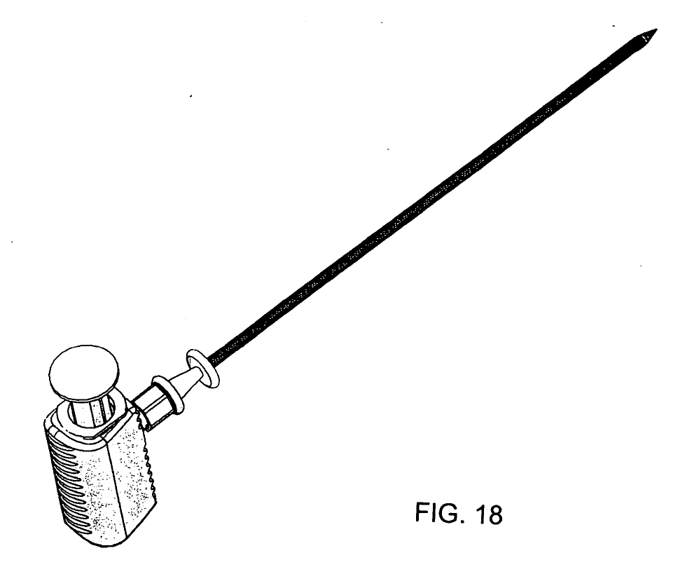

FIGS. 17 and 18 show that the carrier has been slid over the trocar as far as the adjusting nut 7 so that the tip 22 of the trocar 20 protrudes from the opening 44 of the carrier 40. The body 61 of the syringe 60 has been inserted into the bore 10 in the handle 1 and the plunger 65 has been depressed by pushing on the disk 67.

Therefore, a continuous path is formed for lubricant fluid or saline from the syringe 60, through the bore 10, through the channels 11, 12, 13, through the nipple 5 with the nut 7, through the passageway 23 and out of the outlets 25 to flow along the fluted or concave channels 24, 45.

I claim:

1. A percutaneous surgical instrument system, comprising:
a handle to be manipulated by an operator;
a carrier;
a trocar to be manipulated by said handle, inserted through said carrier for penetration to a surgical site, and removed from said carrier to permit insertion of a surgical instrument through said carrier; and
a syringe for supplying fluid to said trocar during penetration to the surgical site.

2. The system according to claim 1, wherein said trocar has a tip through which said syringe supplies the fluid to the surgical site.

3. The system according to claim 2, wherein said tip has at least one opening formed therein for supplying the fluid to the surgical site.

4. The system according to claim 3, wherein said tip has an outer periphery with channels formed therein for directing the fluid, and said at least one opening is disposed in at least one of said channels.

5. The system according to claim 4, wherein said channels extend substantially in axial direction of said trocar and are spaced apart circumferentially entirely over said outer periphery of said tip.

6. The system according to claim 5, wherein said carrier has an outer periphery with channels formed therein for directing the fluid along with said channels formed in said tip of said trocar.

7. The system according to claim 6, wherein said carrier has a widened proximal entrance for receiving said trocar or surgical instrument and a distal opening with a sharpened edge to aid insertion into subject tissue at the surgical site.

8. The system according to claim 1, wherein:
said trocar has a tip with an outer periphery, said outer periphery has channels formed therein, said channels have at least one opening formed therein, and said trocar has a passageway formed therein leading to said at least one opening;
said handle has a bore formed therein for at least partly receiving said syringe, and said handle has at least one passageway formed therein leading to said passageway in said trocar; and
said syringe supplies the fluid through said at least one passageway in said handle and said passageway in said trocar to said at least one opening.

9. The system according to claim 8, wherein said carrier has an outer periphery with channels formed therein for directing the fluid along with said channels formed in said tip of said trocar.

10. The system according to claim 9, wherein said channels in said outer periphery of said tip of said trocar extend substantially in axial direction of said trocar and are spaced apart circumferentially entirely over said outer periphery of said tip.

11. The system according to claim 8, wherein said handle has a threaded nipple and a threaded nut for connecting said at least one passageway in said handle through said nipple to said passageway in said trocar.

12. The system according to claim 8, wherein said syringe has a plunger and a body with a cylindrical container for receiving said plunger, said plunger being manually depressed for injecting the fluid into said handle.

13. The system according to claim 12, wherein said plunger has a head with a recess formed therein and a projection disposed in said recess, forming a ring seal with play allowing the fluid to leak for limiting and controlling injection pressure while adequately lubricating said carrier.

14. A method for percutaneously inserting a surgical instrument to a surgical site, the method comprising the following steps:
inserting a trocar through a carrier;
inserting the carrier with the trocar to the surgical site while guiding a fluid through the trocar;
removing the trocar from the carrier; and
inserting the surgical instrument through the carrier.

15. The method according to claim 14, which further comprises directing the fluid through at least one opening in at least one channel in an outer periphery of a tip of the trocar.

16. The method according to claim 15, wherein the channels extend substantially in axial direction of the trocar and are spaced apart circumferentially entirely over the outer periphery of the tip.

17. The method according to claim 15, which further comprises directing the fluid along the channels formed in the tip of the trocar and along channels formed in an outer periphery of the carrier.

18. The method according to claim 17, which further comprises manipulating the trocar with a handle.

19. The method according to claim 18, which further comprises supplying the fluid from a syringe through the handle and through the trocar to the at least one opening during penetration to the surgical site.

20. The method according to claim 19, which further comprises allowing the fluid to leak out of a ring seal in the syringe for limiting and controlling injection pressure while adequately lubricating the carrier.

21. A percutaneous surgical instrument system, comprising:
a carrier having an outer surface with channels formed therein; and
a trocar to be inserted through said carrier for penetration to a surgical site, and removed from said carrier to permit insertion of a surgical instrument through said carrier;
said trocar having a tip with an outer surface, channels formed in said outer surface and at least one hole in at least one of said channels, for supplying fluid through said at least one hole and along said channels in said carrier and said tip of said trocar.
